(19) United States
(12) Patent Application Publication
(10) Pub. No.: US 2009/0028990 A1
(43) Pub. Date: Jan. 29, 2009

(54) Food composition for stimulating growth comprising fraction isolated from mammalian colostrum or milk whey

(75) Inventors: Suk-Hyung Kwon, Seoul (KR); Jeong-Rai Lee, Chungcheongbuk-do (KR); Hyun-Mi Kim, Chungcheongbuk-do (KR); Kuk-Hwan Kim, Chuncheongbuk-do (KR); Eun-Ju Yoon, Daejeon (KR)

Correspondence Address: BUCHANAN, INGERSOLL & ROONEY PC, POST OFFICE BOX 1404, ALEXANDRIA, VA, 22313-1404, US

(73) Assignee: Rexgenebiotech Co., Ltd, Chungeheongbuk-do (KR)

(21) Appl. No.: 11/920,519

(22) PCT Filed: Aug. 18, 2005
(86) PCT No.: PCT/KR2005/002723

§ 371 Date: Nov. 16, 2007

(30) Foreign Application Priority Data
May 27, 2005 (KR) 10-2005-0044946

(51) Int. Cl. A23L 1/29; A23C 9/14; A61K 35/20

(52) U.S. Cl. 426/2; 426/583; 426/61

(57) ABSTRACT

The present invention relates to a food composition for stimulating growth, comprising a fraction isolated from whey derived from mammalian colostrum or milk. More particularly, the present invention relates to a food composition for stimulating growth, comprising a fraction isolated from mammalian whey, wherein the fraction was removed α-lactoalbumin and β-lactoglobulin from the whey and had a molecular weight of 1-30 kD, as well as a method for stimulating growth, which comprises administering the food composition to mammals in need thereof, and a use of the isolated fraction for preparing the growth-stimulating food composition.

### FIELD OF THE INVENTION

The present invention relates to a food composition for stimulating growth, comprising a fraction isolated from whey derived from mammalian colostrum or milk. More particularly, the present invention relates to a food composition for stimulating growth, comprising a fraction isolated from mammalian whey, wherein the fraction was removed α-lactoalbumin and β-lactoglobulin from the whey and had a molecular weight of 1-30 kD, as well as a method for stimulating growth, which comprises administering the food composition to mammals in need thereof, and a use of the isolated fraction for preparing the growth-stimulating food composition.

### BACKGROUND OF THE INVENTION

Recently, the growth and development of infants and juveniles in Korea are greatly improving due to the continuous growth of economy, the westernization of eating habits and the improvement of nutrition. Also, as a social atmosphere favoring great height has been formed by mass media, most people have an interest in growth.

Growth signifies stature growth according to an increase in the number and size of human body cells. Growth of stature signifies increase in the length of bones and development of muscles around bones, resulted by the explosive differentiation, development and proliferation of osteoblasts in the growth plate, a special tissue existing only in the childhood and adolescence stages. Particularly, this procedure occurs by the action of growth hormones.

Until now, the methods to stimulate growth such as administration of growth hormone preparations, ilizarov surgery, and administration of diet supplements have been used. The administration of growth hormone preparations has excellent effects for people who lack growth hormones. However, in most people with normal hormone secretion, it can cause various side effects, such as acromegaly, growth hormone antibody formation, systemic allergic reactions, and hypothyroidism. Also, the ilizarov surgery, which is surgery of cutting a leg bone and lengthening it is too extreme to use for ordinary people in terms of patient's pain and cost. In addition, the diet supplements for growth stimulation are mostly scientifically unproven.

Whey, which is isolated from mammalian milk, is a liquid byproduct obtained mainly in the preparation of cheese or casein. It is generally known that 85-90% of raw milk is obtained as whey in the preparation of cheese. The production of whey gradually increases owing to the development of the milk processing industry, particularly the cheese industry, but whey is partially used as feed and mostly wasted since its industrial application is low. Recently, as whey is known to contain useful components, such as protein, lactose, minerals and water-soluble vitamins, various studies to use whey are now conducted. Therefore, studies about preparing whey-containing beverages or whey cheese, butter, powder and concentrated whey for use as a raw material in the confectionery or fermentation industry are now being conducted.

Despite such efforts, whey is mostly wasted due to processing limitations.

### DETAILED DESCRIPTION OF THE INVENTION

The present inventors have conducted studies for many years to develop a food having activity capable of effectively stimulating growth without side effects, and as a result, found that a fraction with a molecular weight of 1-30 kD isolated from whey derived from mammalian colostrum or milk has the ability to stimulate growth, thereby completing the present invention.

Therefore, it is an object of the present invention to provide a food composition for stimulating growth, comprising a fraction isolated from mammalian whey.

It is another object of the present invention to provide a method for stimulating growth, which comprises administering, the food composition to a mammal in need thereof.

It is another object of the present invention to provide a use of the fraction isolated from mammalian whey for preparing the food composition for stimulating food composition.

To achieve the above objects, in one aspect, the present invention provides a food composition for stimulating growth, comprising a fraction isolated from mammalian whey.

In another aspect, the present invention provides a method for stimulating growth, comprising administering the food composition to a mammal in need thereof.

In still another aspect, the present invention provides a use of the fraction isolated from mammalian whey for preparing the food composition for stimulating food composition.

Hereinafter, the present invention will be described in detail.

The fraction isolated from whey, which is contained in the food composition according to the present invention, is characterized in that it is isolated from mammalian whey, contains no α-lactoalbumin and β-lactoglobulin and has a molecular weight of 1-30 kD.

Preferably, the fraction isolated from whey, which is contained in the inventive composition, can be prepared by a method comprising the steps of: (a) removing fat and casein from mammalian milk to obtain whey; (b) removing α-lactoalbumin and β-lactoglobulin from the whey of the step (a); and (c) isolating and collecting a fraction with a molecular weight of 1-30 kD from the whey removed α-lactoalbumin and β-lactoglobulin of the step (b).

The mammals in the step (a) include all animals secreting milk, for example, cattle, goats, sheep and human beings. In the present invention, cattle are particularly preferred.

The milk in the step (a) is preferably colostrum. Colostrum refers to a milk component secreted by the mammary gland of mammals up to 3 days after delivery.

Also, the step (a) preferably comprises the sub-steps of: (i) centrifuging the mammalian milk to remove a cream layer so as to obtain skim milk; and (ii) adjusting the pH of the skim milk to 4.0-5.0 of the step (i) to coagulate casein and then centrifuging the skim milk to obtain the supernatant.

The removal of α-lactoalbumin and β-lactoglobulin in the step (b) may be performed by a protein separation method known in the art, and preferably, a separation method using the isoelectric points of proteins. Examples of the separation method using the isoelectric points of proteins include electrophoresis, salting-out, and ion exchange chromatography.

The electrophoresis is a separation and analysis method using the charge of protein molecules. The protein molecules generally bear a positive or negative charge at a pH other than their isoelectric points, and thus, when a direct current is applied to the protein molecules in buffer with a constant pH, the protein molecules will move toward a positive or negative electrode. Namely, the electrophoresis is a method for performing separation and analysis using the fact that the movement rates of protein molecules in an electric field vary depending on the charge and size of the protein molecules.

The salting-out method is a technique of precipitating and separating proteins by adding a salt (e.g., ammonium sulfate), which is well soluble in water, to the protein mixture to increase the ion intensity of the proteins.

The ion exchange chromatography is a technique using a characteristic in that proteins bear a positive or negative charge at a suitable pH. Based on this characteristic, a polymeric electrolyte as filler is placed in a column, and proteins are fractionated according to differences in the ion interaction of the filler with the charge of the proteins.

Preferably, the removal of α-lactoalbumin and β-lactoglobulin in the step (b) can be performed by the salting-out method. More preferably, it can be performed by a salting-out method comprising the steps of: (i) adjusting the pH of the whey removed fat and casein of the step (a), to 5.0-7.0, and then adjusting the pH of the whey to 2.5-4.2, so as to precipitate α-lactoalbumin, and obtaining the supernatant; (ii) adjusting the pH of the supernatant obtained in the step (i) to 4.5-7.0 to precipitate β-lactoglobulin, and obtaining the supernatant.

The adjustment of pH in the salting-out method can be performed by adding a basic or acidic compound. The basic compound is selected from the group consisting of monosodium citrate, disodium citrate, trisodium citrate, sodium hydroxide and potassium hydroxide. Examples of the acidic compound, which can be used in the salting-out method, include, but is not limited thereto. The acid compound is selected from the group consisting of citric acid, hydrochloric acid, sulfuric acid and nitric acid, but is not limited thereto.

In the case where the fraction isolated from the whey is prepared by the salting-out method as described above, α-lactoalbumin and β-lactoglobulin, which have been generally present in the form of monomers in milk, are converted into polymers to form precipitates, which make it easy to remove α-lactoalbumin and β-lactoglobulin from the whey.

The isolation of the fraction with a molecular weight of 1-30 kD in the step (c) can be performed by an isolation method known in the art, which performs isolation according to the molecular weight size of proteins. Preferably, an ultrafiltration or gel filtration method may be used.

Furthermore, the fraction isolated from whey, which is contained in the inventive food composition, may be one obtained by further performing the step of drying and powdering the fraction obtained in the step (c). The drying of the fraction may be carried out by any method in the art, and preferably, a method selected from the group consisting of spray drying, hot air drying and freeze drying.

The isolated fraction prepared by the above-described method has the activity of stimulating growth. More specifically, the fraction has the activities of increasing bodyweight, lengthening the length of a femur, and stimulating the production of serum insulin-like growth factor-I and growth hormones.

The insulin-like growth factors (IGF), which are peptides acting to stimulate growth, are broadly divided into the following two groups: IGF-I and IGF-II. The IGF-I, which is a polypeptide hormone consisting of 70 amino acids, exists in human plasma while mediating the growth stimulating action of growth hormones. Particularly, the IGF-I plays an important role in growth after birth. The IGF-II, which is a polypeptide hormone consisting of 67 amino acids, has a division stimulating effect in a long-term view and a metabolism stimulating effect in a short-term view.

The growth hormones, which are hormones secreted from the anterior pituitary gland, are contained in blood in small amounts and at the same time, converted into factors aiding growth in the liver. Such growth hormones not only stimulate the differentiation of chondrocytes in the growth plate at the end of bones to directly promote the growth of bones, but also act on the liver, the kidneys and the like to stimulate the production of insulin-like growth factors so as to stimulate the absorption of proteins, thus growing muscles, and to stimulate the proliferation of chondrocytes, thus aiding the growth of bones.

In one test example of the present invention, rats in the growth stage were allowed to ingest either a general diet or test diets containing varying concentrations of the inventive isolated fraction and then measured for a change in bodyweight (see Test Example <1-2>) and a change in the length of a femur (see Test Example <1-3>). As a result, white rats ingested with the test diets containing the inventive fraction showed an increase in bodyweight (see Table 2) and an increase in the length of the femur (see Table 3 and FIG. 1), as compared to the control group ingested with the general diet.

Furthermore, in other test examples of the present invention, rats in the growth stage were allowed to ingest either a general diet or test diets containing the inventive fraction and measured for serum IGF-I concentration (see Test Example <1-4>) and growth hormone concentration (see Test Example <1-5>). As a result, rats ingested with the test diet containing the inventive fraction showed increases in serum IGF-I concentration (see Table 4 and FIG. 2) and growth hormone concentration (see Table 5 and FIG. 3), as compared to the control group ingested with the general diet.

From the test results, the present inventors could confirm that the fraction isolated from whey according to the present invention has the activity of stimulating growth.

Accordingly, the present invention provides a food composition for the stimulation of growth, comprising the fraction isolated from whey. Preferably, the food composition according to the present invention is effective in stimulating the growth and bone formation of infants and juveniles in the growth stage.

The food composition according to the present invention may include in all possible forms, such as functional food, nutritional supplement, health food and food additives. These forms of the food compositions can be prepared in various forms according to any conventional method known in the art.

For example, the health food can be prepared and drunk in the form of tea, juice and a drink, or prepared and ingested in the form of paste, granule, tablet and powder.

Also, the fraction isolated from whey can be prepared in the form of powder or concentrated solution and added to food for stimulating the growth of infants and juveniles. For example, the inventive isolated fraction may be added to beverages, fruits and their processed foods (e.g., canned fruit foods, bottled foods, jams, marmalades, etc.), fishes, meats and their processed foods (e.g., hams, sausages and corned beefs), breads and noodles (e.g., Japanese noodles, buckwheat noodles, instant noodles, spaghetti, macaroni, etc.), fruit juice, various drinks, cookies, milk products (e.g., butter, cheese, etc.), edible vegetable oil and fat, margarine, vegetable protein, retort food, frozen food, various seasonings (e.g., soybean paste, soy sauce, sauces, etc.), and the like.

The content of the fraction isolated from whey in the inventive food composition may be 0.01-90% by weight and preferably 0.1-50% by weight, based on the total weight of food, but is not specially limited to any value.

Furthermore, in addition to the fraction isolated from whey, the inventive food composition may further contain trace amounts of minerals, vitamins, lipids, saccharides and known components with growth stimulation activity.

Examples of the minerals, which can be contained in the inventive food composition, include nutrients necessary in the growth stage, such as calcium and iron. Examples of vitamins, which can be contained in the inventive food composition, include vitamin C, vitamin E, vitamin B₁, vitamin B₂, and vitamin B₆. Examples of the lipids, which can be contained in the inventive food composition, include alkoxyglycerol and lecithin. Examples of the saccharides include fructooligosaccharide.

Preferably, the inventive food composition may consist of 0.1-50% by weight of the inventive fraction isolated from whey, 0.5-60% by weight of edible whey calcium, 0.01-30% by weight of alkoxyglycerol powder, 0.1-80% by weight of lecithin powder, 0.01-35% by weight of milk protein hydrolysate powder, 0.01-20% by weight of Chlorellar extract powder, 0.1-50% by weight of fructooligosaccharide, 0.01-35% by weight of heme-iron, 0.001-20% by weight of vitamins, and 0.1-99% by weight of lactose.

The edible whey calcium is obtained by concentrating the mineral components of milk whey, and contains, in addition to calcium, other water-soluble vitamins or nitrogen compounds in a non-protein form, and thus, provides micronutrients together with calcium.

The alkoxyglycerol powder is obtained by extracting shark liver oil and serves as a self defense factor that stimulates human bone marrow cells to promote the production of human immune factors, such as leukocytes and platelets.

The lecithin powder, which is a main component of phospholipid, a kind of complex lipid, is prepared through separation from soybeans or egg yolks. The lecithin forms a body cell membrane, the main function of cholesterol, and is an important fat component which is most frequently used in the body together with neutral fat playing an important role in a cell signaling system.

The milk protein hydrolysate powder is obtained by hydrolyzing milk protein with enzyme or acid to make the milk protein edible and contains casein phosphopeptide (CPP) that stimulates the in vivo absorption of calcium. Thus, the addition of the milk protein hydrolysate powder to the inventive composition can increase the growth stimulating effect of the inventive fraction isolated from whey.

The Chlorellar extract powder is obtained by extracting Chlorellar with hot water, centrifuging the extract to remove the insoluble material of Chlorellar, and extracting and concentrating useful substances, such as proteins, amino acids, saccharides, water-soluble vitamins, inorganic salts, and nucleic acid-related substances. Particularly, the Chlorellar extract contains growth factor “CGF” with the effect of stimulating the growth of children.

The heme iron is obtained by treating bovine or porcine blood with enzyme and the like to isolate a heme protein and purifying the heme protein so as to be edible and is used for the purpose of iron enhancement.

The vitamins may include vitamin C, vitamin E, nicotinic amide, vitamin A, vitamin B₁, vitamin B₆ and biotin, and these vitamins all act to stimulate the absorption of calcium.

The lactose is prepared by separating only a carbohydrate component from skim milk or whey and powdering the isolated carbohydrate.

Moreover, the present invention provides a method for stimulating growth, which comprises administering an effective amount of either the growth-stimulating food composition containing the fraction isolated from whey, or the food composition containing, the isolated fraction from whey, trace amounts of minerals, vitamins, lipids, saccharides and known components with growth stimulating activity, to a mammal in need thereof.

Preferably, the growth stimulation is caused by increases in bodyweight, bone length, intrinsic IGF-I level, intrinsic growth hormone level.

For example, of the mammal used in the present invention include, but are not limited to, human being, cattle, horse, pig, rodent, and sheep. Preferably, the mammals may be human beings, and more preferably, healthy infants and juveniles in the growth stage.

As used herein, the term “effective amount” refers to an amount capable of stimulating the growth of mammals. Preferably, the effective amount is in a range of 0.1 mg/kg bodyweight/day to 1000 mg/kg bodyweight/day.

Also, the administration in the present invention is preferably oral administration.

In another aspect, the present invention provides a use of a fraction isolated from mammalian whey for preparing the food composition for stimulating growth, wherein the fraction is removed α-lactoalbumin and β-lactoglobulin from the whey and has a molecular weight of 1-30 kD.

The fraction isolated from whey may be prepared by the above-described method.

### BRIEF DESCRIPTION OF THE DRAWINGS

FIG. 1 shows measurement results for the mean length of femur of test animals with the ingestion of the inventive fraction isolated from whey.

Test group 1: a group ingested with a diet comprising 0.05% by weight of the fraction isolated from whey;

Test group 2: a group ingested with a diet comprising 0.5% by weight of the fraction isolated from whey; and

Test group 3: a group ingested with a diet comprising 5% by weight of the fraction isolated from whey.

FIG. 2 shows measurement results for the serum IGF-I concentration of test animals with the ingestion of the inventive fraction isolated from whey.

Test group 1: a group ingested with a diet comprising 0.05% by weight of the fraction isolated from whey;

Test group 2: a group ingested with a diet comprising 0.5% by weight of the fraction isolated from whey; and

Test group 3: a group ingested with a diet comprising 5% by weight of the fraction isolated from whey.

FIG. 3 shows measurement results for the serum growth hormone concentration of test animals with the ingestion of the inventive fraction isolated from whey.

Test group 1: a group ingested with a diet comprising 0.05% by weight of the fraction isolated from whey;

Test group 2: a group ingested with a diet comprising 0.5% by weight of the fraction isolated from whey; and

Test group 3: a group ingested with a diet comprising 5% by weight of the fraction isolated from whey.

### EXAMPLES

Hereinafter, the present invention will be described in detail by examples. It is to be understood, however, that these examples are for illustrative purpose only and are not construed to limit the scope of the present invention.

### Example 1

### Preparation of Inventive Fraction Isolated from Whey

Skim colostrum was prepared by collecting colostrum for 48 hours after delivery and centrifuging the collected colostrum to remove the cream layer. The skim colostrum was adjusted to a pH of 4.6 by the addition of 1N hydrochloric acid to coagulate casein and then centrifuged to obtain colostrum whey as the supernatant. The isolated whey was adjusted to a pH of 5.5 by the addition of 1.5M trisodium citrate, and then, to a pH of 3.9 by the addition of 1.5M citric acid. Then, the whey was warmed at 35° C. for 150 minutes to precipitate α-lactoalbumin. The whey was centrifuged to remove α-lactoalbumin, and the supernatant was collected. The supernatant was adjusted to a pH of 4.5 by the addition of 1N NaOH to precipitate β-lactoglobulin and centrifuged to obtain the supernatant. The obtained supernatant was passed through a 30-kD ultrafiltration membrane, and the filtrate was passed through a 1 kD ultrafiltration membrane to obtain a residue. The residue was freeze-dried to obtain a powder fraction with a molecular weight of 1 kD to 30 kD.

### Test Example 1

### Examination of Growth-Stimulating Effect of Fraction Isolated from Whey Using Animal Models

<1-1> Ingestion of Fraction Isolated from Whey

28 male white SD (Sprague-Dawley) rats (three-week-old, weighing 50-65 g) in the growth stage were acclimated for one week, and then, divided into four groups each consisting of 7 animals and used in tests. Raising conditions in the tests were as follows: temperature: 23±1° C.; humidity: 40-60%; and a 12-hr light/12-hr dark cycle. During the acclimation period of 1 week, a general diet and drinking water were provided without limitation, and during a test period of 6 weeks, a diet prepared for each of the groups together with water was provided without limitation. For the control group, a general diet comprising 20% by weight of protein was provided and casein was used as a protein source. Test groups were provided with diets comprising a mixture of the isolated fraction of Example 1 and casein in order to contain the total protein of 20% by weight in the diets. Namely, for ingestion to the test groups, the general diet was mixed with each protein source consisting of: a mixture of 0.05% by weight of the isolated fraction and 19.95% by weight of casein (test group 1); a mixture of 0.5% by weight of the isolated fraction and 19.5% by weight of casein (test group 2); and a mixture of 5% by weight of the isolated fraction and 15% by weight of casein (test group 3). See Table 1 below.

TABLE 1
Composition of test diets

| | Control | | Test | |
|---|---|---|---|---|
| | group | Test group | group 2 | Test group |
| | (wt %) | 1 (wt %) | (wt %) | 3 (wt %) |
| Casein | 20 | 19.95 | 19.5 | 15 |
| Whey-isolated fraction of | — | 0.05 | 0.5 | 5 |
| Example 1 | | | | |
| Corn starch | 39.7486 | 39.7486 | 39.7486 | 39.7486 |
| α-corn starch | 13.2 | 13.2 | 13.2 | 13.2 |
| Sugar | 10 | 10 | 10 | 10 |
| Cellulose | 5 | 5 | 5 | 5 |
| Soybean Oil | 7 | 7 | 7 | 7 |
| Tert-butylhydroquinone | 0.0014 | 0.0014 | 0.0014 | 0.0014 |
| Cystine | 0.3 | 0.3 | 0.3 | 0.3 |
| Choline hydrogen tartrate | 0.25 | 0.25 | 0.25 | 0.25 |
| Vitamin mix¹⁾ | 1 | 1 | 1 | 1 |
| Mineral mix²⁾ | 3.5 | 3.5 | 3.5 | 3.5 |
| Total | 100 | 100 | 100 | 100 |

¹⁾210025 AIN-93G mineral mix
²⁾310025 AIN-VX vitamin mix

<1-2> Changes in Bodyweight of Test Animals with Ingestion of Fraction Isolated from Whey

Each of the animal groups in Test Example <1-1> was measured for bodyweight before ingestion of the diets and bodyweight after 6 weeks of ingestion of the diets. The measured values are statistically processed to calculate mean values and standard deviations, and the measured bodyweight was used to calculate an increase in bodyweight according to the following equation:

Increase in mean bodyweight=(mean bodyweight at the end of test−mean bodyweight at the start of test)

To test the significance between the control group and each of the test groups, the data was statistically processed with Student's t-test, and then, when a confidence interval (P value) was lower than 0.05, it would be determined to be statistically significant.

In the test results, the test groups ingested with the inventive fraction isolated from whey all showed an increase in bodyweight as compared to the control group. Particularly, the test group 3 ingested with the diet comprising 5% by weight of the isolated fraction showed a significant increase in bodyweight as compared to the control group (see Table 2).

TABLE 2
Changes in bodyweight of test animals with ingestion of fraction isolated from whey

| | | Group administered | Groups administered with fraction | | |
|---|---|---|---|---|---|
| | | with general diet | isolated from whey | | |
| | | Control group 1 | Test group 1 | Test group 2 | Test group 3 |
| Mean | Before | 58.1 ± 2.79¹⁾ | 57.1 ± 2.19 | 57.6 ± 2.56 | 55.6 ± 0.90 |
| body | administration of | | | | |
| weight | diet | | | | |
| | After | 358.4 ± 31.78 | 382.4 ± 20.57 | 388.3 ± 26.90 | 406.0 ± 27.11 |
| | administration of | | | | |
| | diet | | | | |
| | Increase in | 300.3 ± 30.34 | 325.3 ± 20.48 | 330.7 ± 27.34 | 350.4 ± 27.71** |
| | bodyweight | | | | |

¹⁾mean ± standard deviation (n = 7)
**significant difference at P < 0.01

<1-3> Measurement of Change in Femur Length of Test Animals with Ingestion of Fraction Isolated from Whey

After completion of the 6-week tests in Example <1-1>, the left and right femurs of the test groups ingested with the inventive isolated fraction and the control group were taken out. Muscles, fats, ligaments and the like adhered to the femur tissue were completely removed, and the femurs were dried in a drying oven at 75° C. for 3 hours and then measured for their length.

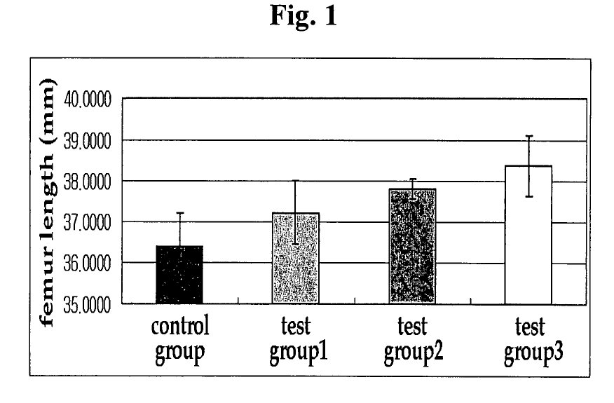

In the test results, the test groups ingested with the inventive fraction isolated from whey showed a dose-dependent increase in femur length as compared to the control group. Particularly, the test groups 2 and 3 ingested with the diets containing the isolated fraction in amounts of 0.5% by weight and 5% by weight, respectively, showed a significant increase in femur length as compared to the control group (see Table 3 and FIG. 1).

TABLE 3
Femur length of test animals with ingestion of fraction
isolated from whey

| | | Left femur | Right femur | Mean femur |
|---|---|---|---|---|
| Groups | | length (cm) | length (cm) | length (mm) |
| Control group | | 36.39 ± 0.83¹⁾ | 37.37 ± 0.32 | 36.88 ± 0.48 |
| Test groups | Test | 37.22 ± 0.78 | 37.35 ± 0.92 | 37.28 ± 0.84 |
| | group 1 | | | |
| | Test | 37.80 ± 0.24 | 38.31 ± 0.45** | 38.05 ± 0.30** |
| | group 2 | | | |
| | Test | 38.37 ± 0.75** | 38.35 ± 0.67* | 38.37 ± 0.64** |
| | group 3 | | | |

¹⁾: mean ± standard deviation (n = 7)
*significant difference at P < 0.05
**significant difference at P < 0.01

<1-4> Measurement of Serum IGF-I Concentration of Test Animals with Ingestion of Fraction Isolated from Whey

In order to examine if the inventive fraction isolated from whey has the effect of the secretion of IGF-I to stimulate growth, the following test was performed. After completion of the 6-week tests in Test Example <1-1>, the blood samples of the control group and the test groups were collected and measured for serum IGF-I concentration using OCTEIA Rat IGF-I kit (IDS Co., USA).

Described specifically, 25 μl of serum and 100 μl of a releasing reagent were placed in a test tube and allowed to react with each other at 18-28° C. for 10 minutes, and then, mixed with 1 ml of PBS (phosphate-buffered saline) comprising 0.05% sodium azide. Then, 25 μl of each of the mixtures was placed in a 96-well microplate, to which 100 μl of anti-rat IGF-I biotin was added. Then, the mixture and the biotin were allowed to react with each other in a microplate shaker (500-700 rpm) at 18-28° C. for 2 hours. After completion of the reaction, each well was washed three times and added with 200 μl of an enzyme conjugate (horseradish peroxidase), and the mixture was allowed to react at 18-28° C. for 30 minutes, and then washed three times. Next, each well was added with 200 μl of TMB (tetramethylbenzidine) as a substrate, and the mixture in each well was allowed to react at 18-28° C. for 30 minutes. Then, 100 μl of HCl was added to the reaction mixture to stop the reaction. Then, the mixture was measured for the absorbance at 450 nm using a microplate reader (ELx800, Bio-tec, USA).

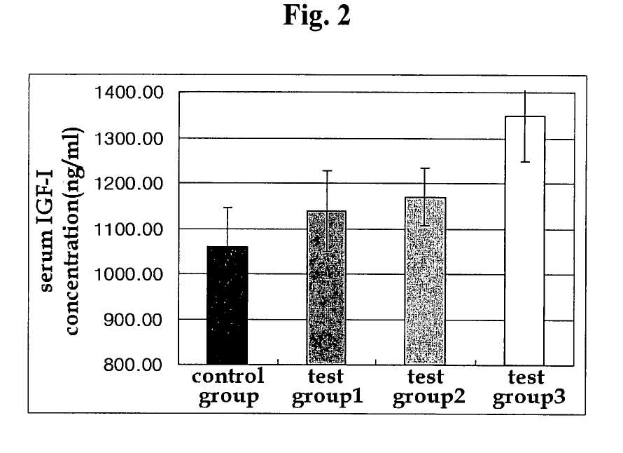

In the test results, the test groups ingested with the inventive fraction isolated from whey showed an increase in serum IGF-I concentration in a dose-dependent manner as compared to the control group. Particularly, the test groups ingested with the diets comprising the inventive isolated fraction in amounts of 0.5% by weight and 5% by weight, respectively, showed a significant increase in serum IGF-I concentration as compared to the control group (see Table 4 and FIG. 2).

TABLE 4
Serum IGF-I concentration of test animals with ingestion
of fraction isolated from whey

| Groups | | Serum IGF-I concentration (ng/ml) |
|---|---|---|
| Control group | | 1060.29 ± 85.43¹⁾ |
| Test groups | Test group 1 | 1138.54 ± 89.58 |
| | Test group 2 | 1168.62 ± 63.04* |
| | Test group 3 | 1349.10 ± 103.29** |

¹⁾mean ± standard deviation (n = 7)
*significant difference at P < 0.05
**significant difference at P < 0.01

<1-5> Measurement of Serum Growth Hormone Concentration of Test Animals with Ingestion of Fraction Isolated from Whey

In order to examine if the inventive fraction isolated from whey has the effect of stimulating the secretion of growth hormones to stimulate growth; the following test was performed. After completion of the 6-week tests in Test Example <1-1>, the blood samples of the test groups and the control group were collected and measured for serum growth hormone concentration by IRMA (immunoradiometric assay) using Daiichi GH kit (Daiichi Radioisotope Labs., Ltd. Japan).

Described specifically, 50 μl of each of the blood samples was placed in an antibody-coated tube, to which 200 μl of anti-hGH was added. Then, the tube was covered with a cover, and the mixture was allowed to react in a microplate shaker (180 rpm) at room temperature for 4 hours. Then, the supernatant was removed, after which the reaction mixture was washed three times with distilled water. Then, the mixture was measured for serum growth hormone concentration for 1 minute using a γ-counter (COBRA 5010, PACKARD, USA).

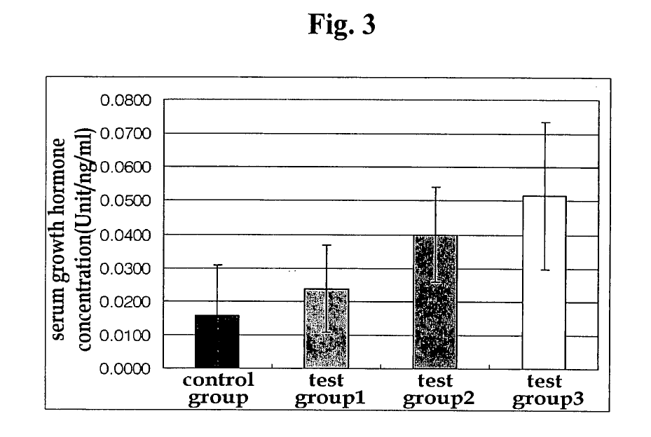

In the test results, the test groups ingested with the inventive fraction isolated from whey showed a dose-dependent increase in serum growth hormone concentration as compared to the control group. Particularly, the test group ingested with the isolated fraction in an amount of 5% by weight showed a significant increase in serum growth hormone concentration as compared to the control group (see Table 5 and FIG. 3).

TABLE 5
Serum growth hormone concentration of test animals with ingestion of
fraction isolated from whey

| | | Serum growth hormone |
|---|---|---|
| Groups | | concentrations (ng/ml) |
| Control group | | 0.0157 ± 0.0151¹⁾ |
| Test groups | Test group 1 | 0.0238 ± 0.0130 |
| | Test group 2 | 0.0400 ± 0.0141 |
| | Test group 3 | 0.0514 ± 0.0219** |

¹⁾mean ± standard deviation (n = 7)
**significant difference at P < 0.01

### Example 2

### Preparation of Growth-Stimulating Food Composition Comprising Fraction Isolated from Whey

The growth-stimulating food composition comprising the inventive isolated fraction prepared in Example 1 was prepared by blending components shown in Table 6, and tableted and coated according to a conventional method, thus preparing tablets.

TABLE 6
Components and contents of growth-stimulating food composition
comprising fraction isolated from whey

| Components | Contents (%) |
|---|---|
| Powder fraction isolated from whey | 5.00 |
| Edible whey calcium (more than 25% of calcium) | 20.00 |
| Alkoxyglycerol powder | 3.00 |
| Lecithin powder | 2.00 |
| Milk protein hydrolysate powder | 2.00 |
| Chlorellar extract powder | 0.50 |
| Fructooligosaccharide | 2.30 |
| Heme iron | 0.30 |
| Vitamin C | 0.50 |
| Powder vitamin E (50% dl-α-tocopherol acetate) | 0.50 |
| Nicotinic amide | 0.05 |
| Powder vitamin A | 0.10 |
| Vitamin B₂ | 0.03 |
| Vitamin B₁ nitrate | 0.03 |
| Vitamin B₆ hydrochloride | 0.03 |
| Biotin | 0.01 |
| Lactose | 63.65 |
| Total | 100.00 |

### INDUSTRIAL APPLICABILITY

As described above, the inventive fraction isolated from whey has activities of increasing bodyweight, lengthening the length of a femur and stimulating the production of insulin-like growth factor-I and growth hormones. Thus, the food composition comprising the fraction isolated from whey can stimulate the growth of infants and juveniles in the growth stage.

1. A food composition for stimulating growth, comprising a fraction isolated from mammalian whey, wherein the fraction is removed α-lactoalbumin and β-lactoglobulin from the whey and has a molecular weight of 1-30 kD.

2. The food composition of claim 1, wherein the fraction isolated from whey is prepared by a method comprising the steps of:
(a) removing fat and casein from mammalian milk to obtain whey;
(b) removing α-lactoalbumin and β-lactoglobulin from the whey of the step (a); and
(c) isolating and obtaining a fraction with a molecular weight of 1-30 kD from the whey removed α-lactoalbumin and β-lactoglobulin of the step (b)

3. The food composition of claim 2, wherein the step (a) comprises the sub-steps of:
(i) centrifuging the mammalian milk to remove a cream layer so as to obtain skim milk; and
(ii) adjusting the pH of the skim milk of the step (i) to 4-5 to coagulate casein and then centrifuging the skim milk to obtain the supernatant.

4. The food composition of claim 2, wherein the mammalian milk is bovine colostrum.

5. The food composition of claim 2, wherein removing α-lactoalbumin and β-lactoglobulin of step (b) is performed by a method selected from the group consisting of electrophoresis, salting-out, and ion exchange chromatography.

6. The food composition of claim 2, wherein removing α-lactoalbumin and β-lactoglobulin of step (b) is performed by a salting-out method comprising the steps of:
(i) adjusting the pH of the whey removed fat and casein of the step (a), to 5.0-7.0 and then adjusting the pH of the whey to 2.5-4.2, so as to precipitate α-lactoalbumin and obtaining the supernatant; and
(ii) adjusting the pH of the supernatant of the step (i) to 4.5-7.0 to precipitate β-lactoglobulin and obtaining the supernatant.

7. The food composition of claim 2, wherein the isolation of the fraction with a molecular weight of 1-30 kD in the step (c) is performed by an ultrafiltration or gel filtration method.

8. The food composition of claim 2, wherein the method further comprises the step of drying and powdering the fraction with a molecular weight of 1-30 kD, obtained in the step (c).

9. A food composition for stimulating growth of the fraction isolated from whey of claim 1, consisting of 0.1-50% by weight, 0.5-60% by weight of edible whey calcium, 0.01-30% by weight of alkoxyglycerol powder, 0.1-80% by weight of lecithin powder, 0.01-35% by weight of milk protein hydrolysate powder, 0.01-20% by weight of Chlorellar extract powder, 0.1-50% by weight of fructooligosaccharide, 0.01-35% by weight of heme-iron, 0.001-20% by weight of vitamins, and 0.1-99% by weight of lactose.

10. A method for stimulating growth, which comprises administering an effective amount of the food composition of claim 1 to a mammal in need thereof.

11. The method of claim 10, wherein the stimulating growth is caused by increases in bodyweight, bone length, intrinsic IGF-I level, and intrinsic growth hormone level.

12. The method of claim 10, wherein the administering is oral administering.

13. The method of claim 10, wherein the mammal is human being.

14. A method for preparing the food composition for stimulating growth as set forth in claim 1, comprising utilizing a fraction isolated from mammalian whey, wherein the fraction is removed α-lactoalbumin and β-lactoglobulin from the whey and has a molecular weight of 1-30 kD.
